(19) United States
(12) Patent Application Publication
(10) Pub. No.: US 2022/0299353 A1
(43) Pub. Date: Sep. 22, 2022

(54) LEVEL MEASUREMENT APPARATUS

(71) Applicant: JOHNSON MATTHEY PUBLIC LIMITED COMPANY, London (GB)

(72) Inventors: Graham BARKER, Billingham, Cleveland (GB); Oliver John BECKETT, Billingham, Cleveland (GB); Carl TIPTON, Billingham, Cleveland (GB)

(21) Appl. No.: 17/754,190

(22) PCT Filed: Sep. 30, 2020
(86) PCT No.: PCT/GB2020/052370

§ 371 Date: Mar. 25, 2022

(30) Foreign Application Priority Data
Nov. 19, 2019 (GB) 1916827.7

(51) Int. Cl. G01F 23/284; G01F 23/04; H01Q 1/22; G01N 33/28

(52) U.S. Cl. CPC G01F 23/284; G01F 23/04; H01Q 1/225; G01N 33/2847

(57) ABSTRACT

An apparatus for determining the identity, location or level of one or more material phases or the location of an interface between two material phases within a vessel, the apparatus having a transmitter for transmitting an electromagnetic transmission signal, a receiver for receiving an electromagnetic return signal, and an enclosure configured to be at least partially submerged within the one or more material phases in the vessel, the enclosure comprising a wall defining an interior and an exterior of the apparatus to seal the apparatus from ingress of the one or more material phases into the apparatus when submerged within the one or more material phases in the vessel, the enclosure wall being non-transparent to the electromagnetic transmission signal.

### FIELD

The present invention relates to an apparatus for determining the identity, location or level of one or more material phases or the location of an interface between two material phases within a vessel such as an oil separator unit.

### BACKGROUND

The measurement of levels of fill, particularly of fluids including liquids, gases and multi-phase materials such as emulsions and slurries, has been carried out for many years using nucleonic level gauges, by measuring the amount of radiation emitted by a radiation-source which is detected at one or more levels within the vessel. The radiation is attenuated as is passes through materials, the amounts of attenuation being related to the density of the materials between a source and a detector. By comparing the attenuation of radiation detected at different levels of the vessel, it is possible to estimate the height of materials contained in the vessel.

A density profiler based on these principles has been described in WO2000/022387. The device comprises a linear array of sources of ionising radiation which emit radiation towards detectors disposed in one or more linear arrays. When the source array and detector array(s) are positioned so that they traverse the interfaces between two or more fluids in a vessel, the interfaces of the fluids may be identified from the differences in radiation received by each detector in the array. These devices has been successfully deployed for use in storage tanks and oil separators.

It may be undesirable to use a device which embodies a source of ionising radiation. In some parts of the world nucleonic technology may not be a viable option. Alternative detector arrangements with similar functionality that do not require a source of ionising radiation have accordingly been proposed.

Radar level gauge systems are known for measuring fluid levels in vessels. In particular, guided wave radar level sensor probes are known in which transmitted electromagnetic signals are guided towards and into the vessel by a wave guide, typically arranged vertically from top to bottom of the vessel. The electromagnetic signals are reflected at a fluid surface and received back at the level gauge system by a receiver. The time from emission to reception of the signals is used to determine the level in the vessel.

However, traditional guided wave radar solutions have limitations. For example, while guided wave solutions can detect a clean oil-water interface, they cannot detect an oil-water interface if there is an emulsion in the way. Furthermore, microwaves don't transmit through water and so don't probe effectively beyond a water interface.

It is an aim of the invention to provide a non-nucleonic measurement instrument for measuring levels of materials, especially of fluids, and optionally for measuring/calculating a level profile of a multi-layer fluid column, that mitigates some or all of the foregoing disadvantages of current guided wave radar solutions and/or offers an alternative functionality and/or enhanced accuracy.

### SUMMARY OF THE INVENTION

The present specification provides an apparatus for determining the identity, location or level of one or more material phases or the location of an interface between two material phases within a vessel, the apparatus comprising:
a transmitter for transmitting an electromagnetic transmission signal;
a receiver for receiving an electromagnetic return signal; and
an enclosure configured to be at least partially submerged within the one or more material phases in the vessel, the enclosure comprising a wall defining an interior and an exterior of the apparatus to seal the apparatus from ingress of the one or more material phases into the apparatus when submerged within the one or more material phases in the vessel, the enclosure wall being non-transparent to the electromagnetic transmission signal;
wherein the enclosure comprises at least one window in the enclosure wall, the window being at least partially transparent to the electromagnetic transmission signal, the transmitter arranged to transmit the electromagnetic transmission signal through the window to interact with the one or more material phases outside the window in the vessel, and the receiver arranged to receive the electromagnetic return signal from the window for processing to determine the identity, location or level of the one or more material phases or the location of the interface between two material phases within the vessel,
wherein the window is mounted in the wall of the enclosure with a pressure rating of at least 1000 kPa (10 bar) and a temperature rating of at least 150° C. so as to prevent failure of the window and ingress of the one or more material phases into the apparatus at elevated pressures and temperatures, and
wherein the transmitter is configured to transmit a microwave signal or a radio wave signal.

The enclosure can be in the form of an elongate dip pipe with a plurality of windows provided along the dip pipe. The windows can be directly embedded in the wall of the dip pipe. Alternatively, the windows can be mounted into an elongate window mounting and the elongate window mounting is embedded in the wall of the dip pipe. The enclosure provides a sealed environment in which the electromagnetic signal can be directed to any level of a multi-layered fluid column. As such, the apparatus doesn't have the limitations of traditional guided wave radar solutions in which the electromagnetic radiation is directed down through a fluid column such that reliable detection of lower layers in the multi-layer fluid column is impeded by upper layers in the fluid column.

In one configuration, the apparatus comprises an array of the transmitters and receivers disposed within the elongate dip pipe such that each window has an associated transmitter and receiver.

In an alternative configuration, the apparatus comprises an elongate electromagnetic radiation guide coupled to the transmitter to guide the electromagnetic transmission signal from the transmitter to the plurality of windows along the dip pipe. In this case, the elongate electromagnetic radiation guide can also be configured to guide the electromagnetic return signal back from the plurality of windows to the detector.

The apparatus as described herein must seal the interior of the apparatus from the surrounding material phases in the vessel in which it is disposed. As such, the windows provided in the apparatus enclosure wall must be configured to withstand elevated temperatures and pressures which are experienced in certain vessels which require monitoring using the apparatus. For certain applications, the windows may be configured to have a pressure rating of at least 2000 kPa (20 bar), 3000 kPa (30 bar), 4000 kPa (40 bar), or 5000 kPa (50 bar) and/or a temperature rating of at least 200° C., 250° C., or 300° C. The windows may be configured to have a pressure rating up to 10,000 kPa (100 bar) and a temperature rating up to 500° C. for example.

In order to increase the pressure rating of the windows they may be mounted in the wall of the enclosure via a tapered seal, in the form of a sloped or stepped wall seal, to prevent the windows from being pushed into the apparatus when subjected to pressure from the material phases within the vessel in which the apparatus is disposed. In certain configurations, the enclosure wall is metallic, the one or more windows are glass, and the one or more glass windows are mounted in the metallic wall of the enclosure via a glass-metal bond. Sight glasses, such as borosilicate sight glasses, are available which are pressure rated to high temperatures. The windows can be mounted by heating the metal surround to expand the surround, inserting the windows, and allowing the surround to cool and contract around the windows. The edge of the windows melts and bond to the metal surround via a glass-metal bond and the surround contracts on cooling to compress the window providing a tight pressure seal. This mounting method also has the advantage of avoiding the use of adhesives which could be reactive with materials within a vessel in which the apparatus is disposed in use. That is, the glass-metal bond provides a chemically inert seal.

The apparatus as described herein is used to determining the identity, location or level of one or more material phases or the location of an interface between two material phases within a vessel. A method of determining the identity, location or level of one or more material phases or the location of an interface between two material phases within a vessel is provided, the method comprising:
introducing the apparatus into a vessel;
transmitting an electromagnetic transmission signal through the one or more windows, the electromagnetic transmission signal being a microwave signal or a radio wave signal;
receiving an electromagnetic return signal from the one or more windows; and
   processing the electromagnetic return signal to determining the identity, location or level of one or more material phases or the location of an interface between two material phases within the vessel.

### BRIEF DESCRIPTION OF THE DRAWINGS

The invention will now be described by way of example only with reference to the accompanying drawings, in which:

FIG. 1 shows a cross-section through a level measurement apparatus comprising a plurality of microwave transceivers;

FIG. 2 shows a cutaway schematic of another level measurement apparatus including a microwave transceiver and a waveguide;

FIG. 3 is a schematic depiction of an oil-water separator including a level measurement apparatus;

FIG. 4 shows a dip pipe for use with the apparatus shown in FIGS. 1 and 2, where a plurality of windows have been mounted in an elongate window mounting and the elongate window mounting is embedded in the wall of the dip; and

FIG. 5 a dip pipe for use with the apparatus shown in FIGS. 1 and 2, where a plurality of windows have been directly mounted into the dip pipe.

### DETAILED DESCRIPTION

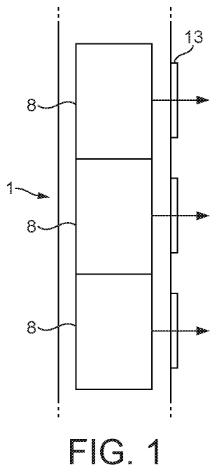

FIG. 1 depicts a schematic cross-sectional view of a level measurement apparatus comprising a plurality of microwave transceivers 8 within an enclosure 12. Also depicted are windows 13 which are located to allow electromagnetic radiation to pass out of and back into the enclosure 12. When used in fluid environments the enclosure 12 may be a dip tube or dip pipe that provides a mechanical and chemical resistant barrier between the microwave transceivers and the materials being profiled. The material of the enclosure is chosen to have sufficient strength and chemical resistance.

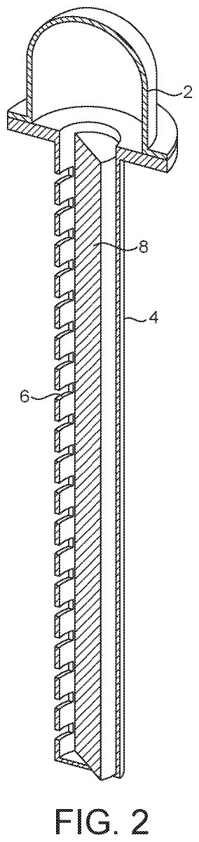

In the arrangement shown in FIG. 1, each window 13 is associated with a corresponding microwave transceiver 8. FIG. 2 shows an alternative arrangement which has a microwave transceiver in a housing 2 and an elongate probe portion 4 which extends into the vessel and contents in use. The probe portion 4 comprises an elongate cylindrical microwave wave guide defined by a conductive wave guide wall. The wave guide couples radiatively to the emitter to act to transfer emitted electromagnetic radiation along its length. A longitudinally spaced array of wave guide slots 6 is provided in the conductive waveguide wall. When the waveguide probe 4 is inserted into a vessel containing multiple layers of substances from which profile information is required, the return signal produced by the signal from each slot 6 in the waveguide after an interaction with the material at the respective slot (for example transmission and/or reflection) can be studied to gain an understanding of the dielectric properties of the material at the respective slots, and from this profile information can be inferred.

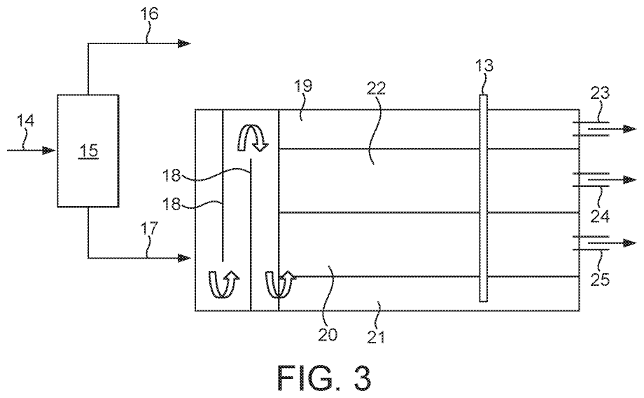

FIG. 3 is a schematic depiction of the level measurement apparatus located within an oil-water separator. The enclosure 13 is shown as arranged in a vertical array that extends substantially the whole height of the separator. The enclosure 13 passes through a wall of the separator vessel and is immersed in the material layers within the vessel. The input flow 14 is a mixture of oil, gas, and water which is passed into a pre-treater 15 to effect preliminary separation of gas which is taken off via line 16, usually for further processing. Liquids, namely oil and water are taken off via line 17. The fluid flow is slowed and rendered less turbulent by baffles 18 before separating into layers of gas 19, water 20, oil 22, and sand or sediment 21. The separate layers flow out of the vessel through respective ports 23, 24, 25. A further port may be provided to remove sand or sediment 21. In operation, the signal detected by the detector(s) within the enclosure 13 is processed to determine the nature of the material at each window location and thus the locations and depths of each of the layers can be determined throughout the separator. It is also possible to determine the presence, location and thickness of any undesirable mixed layers between the gas and water, and between the water and oil layers.

The instruments illustrated in FIGS. 1 and 2 are capable of profiling complex multi-layered fluid columns including oil/water interfaces and emulsions which may be found in an oil separator unit. As such, the instruments can provide a functional improvement over prior art radar level gauge systems, while also avoiding the use of nucleonic sources. One reason for the improved functionality is that the electromagnetic radiation is not directed through the fluid layers from above. Rather, the electromagnetic radiation is guided through the waveguide and only interacts with the fluid external to the elongate electromagnetic waveguide at defined vertical locations where the windows are provided in the waveguide. In this respect, the configurations are analogous to the provision of multiple nucleonic sources at defined vertical locations. Multiple transmitters can be disposed at varying depths of the fluid column as shows in the configuration of FIG. 1. Alternatively, a waveguide as shown in FIG. 2 can direct electromagnetic radiation from a single transmitter along the elongate waveguide and the windows function to provide multiple interrogation points without the requirement of having individual transmitters. Similarly, the configuration of FIG. 2 does not require multiple receivers disposed at varying depths of the fluid column. The waveguide directs return signals from the plurality of windows along the waveguide such that a single receiver can be provided. In either of the configurations shown in FIGS. 1 and 2, the apparatus must be configured such that fluid does not enter the elongate electromagnetic radiation guide through the windows when introduced into a fluid column.

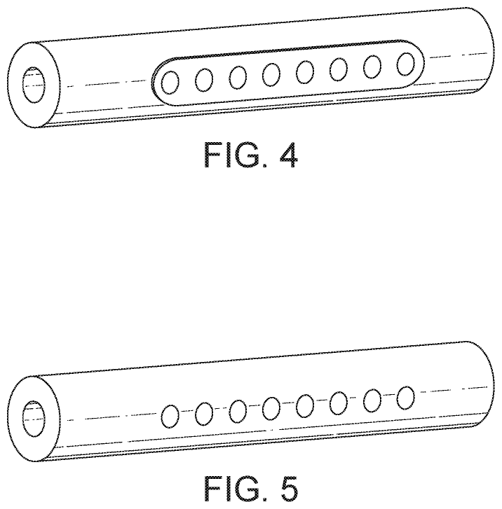

FIGS. 4 and 5 show examples of dip pipes in which a plurality of windows have been mounted. In FIG. 4, the windows have been mounted in an elongate window mounting and the elongate window mounting is embedded in the wall of the dip pipe. In FIG. 5 the windows are mounted directly in the dip pipe. Such dip pipes provide an enclosure configured to be at least partially submerged within the one or more material phases in a vessel, the enclosure comprising a wall defining an interior and an exterior of the apparatus to seal the apparatus from ingress of the one or more material phases into the apparatus when submerged within the one or more material phases in the vessel. The dip pipe wall is non-transparent to electromagnetic transmission signals. In contrast, the windows are at least partially transparent to electromagnetic transmission signals.

One or more transmitters are arranged to transmit electromagnetic transmission signals through the windows to interact with the one or more material phases outside the windows in the vessel. Receivers are arranged to receive electromagnetic return signals from the windows and process the electromagnetic return signals to determining the identity, location or level of the one or more material phases or the location of the interface between two material phases within the vessel.

The windows are mounted in the wall of the dip pipe with a pressure rating of at least 1000 kPa (10 bar) and a temperature rating of at least 150° C. so as to prevent failure of the window and ingress of the one or more material phases into the apparatus at elevated pressures and temperatures. The apparatus as described herein must seal the interior of the apparatus from the surrounding material phases in the vessel in which it is disposed. As such, the windows provided in the apparatus enclosure wall must be configured to withstand elevated temperatures and pressures which are experienced in certain vessels which require monitoring using the apparatus. For certain applications, the windows may be configured to have a pressure rating of at least 2000 kPa (20 bar), 3000 kPa (30 bar), 4000 kPa (40 bar), or 5000 kPa (50 bar) and/or a temperature rating of at least 200° C., 250° C., or 300° C. The windows may be configured to have a pressure rating up to 10,000 kPa (100 bar) and a temperature rating up to 500° C. for example.

The dip pipe provides a sealed environment in which the electromagnetic signal can be directed to any level of a multi-layered fluid column. As such, the apparatus doesn't have the limitations of traditional guided wave radar solutions in which the electromagnetic radiation is directed down through a fluid column such that reliable detection of lower layers in the multi-layer fluid column is impeded by upper layers in the fluid column.

In order to increase the pressure rating of the windows they may be mounted in the wall of the enclosure via a tapered seal, in the form of a sloped or stepped wall seal, to prevent the windows from being pushed into the apparatus when subjected to pressure from the material phases within the vessel in which the apparatus is disposed. In certain configurations, the enclosure wall is metallic, the one or more windows are glass, and the one or more glass windows are mounted in the metallic wall of the enclosure via a glass-metal bond. Sight glasses, such as borosilicate sight glasses, are available which are pressure rated to high temperatures. The windows can be mounted by heating the metal surround to expand the surround, inserting the windows, and allowing the surround to cool and contract around the windows. The edge of the windows melts and bond to the metal surround via a glass-metal bond and the surround contracts on cooling to compress the window providing a tight pressure seal. This mounting method also has the advantage of avoiding the use of adhesives which could be reactive with materials within a vessel in which the apparatus is disposed in use. That is, the glass-metal bond provides a chemically inert seal.

The transmitter is configured to transmit a microwave signal or a radio wave signal. As such, the windows are not required to be transparent to visible light and in certain applications is can be advantageous for the windows to be coloured or opaque to visible light.

The apparatus as described herein is used to determining the identity, location or level of one or more material phases or the location of an interface between two material phases within a vessel. A method of determining the identity, location or level of one or more material phases or the location of an interface between two material phases within a vessel is provided, the method comprising:
introducing the apparatus according to any preceding claim into the vessel;
transmitting an electromagnetic transmission signal through the one or more windows;
receiving an electromagnetic return signal from the one or more windows; and
   processing the electromagnetic return signal to determining the identity, location or level of one or more material phases or the location of an interface between two material phases within the vessel.

For very high-pressure applications, the apparatus may be provided with a pressure control mechanism to alter the pressure within the enclosure and thus reduce the pressure differential across the window(s) in the enclosure.

While this invention has been particularly shown and described with reference to certain embodiments, it will be understood to those skilled in the art that various changes in form and detail may be made without departing from the scope of the invention as defined by the appended claims.

1-14. (canceled)

15. An apparatus for determining the identity, location or level of one or more material phases or the location of an interface between two material phases within a vessel, the apparatus comprising:
a transmitter for transmitting an electromagnetic transmission signal;
a receiver for receiving an electromagnetic return signal; and
an enclosure configured to be at least partially submerged within the one or more material phases in the vessel, the enclosure comprising a wall defining an interior and an exterior of the apparatus to seal the apparatus from ingress of the one or more material phases into the apparatus when submerged within the one or more material phases in the vessel, the enclosure wall being non-transparent to the electromagnetic transmission signal;
wherein the enclosure comprises at least one window in the enclosure wall, the window being at least partially transparent to the electromagnetic transmission signal, the transmitter arranged to transmit the electromagnetic transmission signal through the window to interact with the one or more material phases outside the window in the vessel, and the receiver arranged to receive the electromagnetic return signal from the window for processing to determine the identity, location or level of the one or more material phases or the location of the interface between two material phases within the vessel,
wherein the window is mounted in the wall of the enclosure with a pressure rating of at least 1000 kPa (10 bar) and a temperature rating of at least 150° C. so as to prevent failure of the window and ingress of the one or more material phases into the apparatus at elevated pressures and temperatures, and
wherein the transmitter is configured to transmit a microwave signal or a radio wave signal.

16. The apparatus according to claim 15,
wherein the enclosure is in the form of an elongate dip pipe and a plurality of windows are provided along the dip pipe.

17. The apparatus according to claim 16,
wherein the windows are directly embedded in the wall of the dip pipe.

18. The apparatus according to claim 16,
wherein the windows are mounted into an elongate window mounting and the elongate window mounting is embedded in the wall of the dip pipe.

19. The apparatus according to claim 16,
wherein the apparatus comprises an array of the transmitters and receivers disposed within the elongate dip pipe such that each window has an associated transmitter and receiver.

20. The apparatus according to claim 16,
wherein the apparatus comprises an elongate electromagnetic radiation guide coupled to the transmitter to guide the electromagnetic transmission signal from the transmitter to the plurality of windows along the dip pipe.

21. The apparatus according to claim 20,
wherein the elongate electromagnetic radiation guide is configured to guide the electromagnetic return signal from the plurality of windows to the detector.

22. The apparatus according to claim 15,
wherein the one or more windows have a pressure rating of at least 2000 kPa (20 bar), 3000 kPa (30 bar), 4000 kPa (40 bar), or 5000 kPa (50 bar).

23. The apparatus according to claim 15,
wherein the one or more windows have a temperature rating of at least 200° C., 250° C., or 300° C.

24. The apparatus according to claim 15,
wherein the one or more windows are mounted in the wall of the enclosure via a tapered seal, in the form of a sloped or stepped wall seal, to prevent the one or more windows from being pushed into the apparatus when subjected to pressure from the one or more material phases within the vessel.

25. The apparatus according to claim 15,
wherein the enclosure wall is metallic, the one or more windows are glass, and the one or more glass windows are mounted in the metallic wall of the enclosure via a glass-metal bond.

26. The apparatus according to claim 15,
wherein the one or more windows are not transparent to visible light.

27. Use of the apparatus according to claim 15 to determining the identity, location or level of one or more material phases or the location of an interface between two material phases within a vessel.

28. A method of determining the identity, location or level of one or more material phases or the location of an interface between two material phases within a vessel, the method comprising:
introducing the apparatus according to claim 1 into the vessel;
transmitting an electromagnetic transmission signal through the one or more windows, the electromagnetic transmission signal being a microwave signal or a radio wave signal;
receiving an electromagnetic return signal from the one or more windows; and
processing the electromagnetic return signal to determining the identity, location or level of one or more material phases or the location of an interface between two material phases within the vessel.
